(19) United States
(12) Patent Application Publication
(10) Pub. No.: US 2020/0164123 A1
(43) Pub. Date: May 28, 2020

(54) CHEST TUBE VALVE

(71) Applicants: Chris Salvino, Scottsdale, AZ (US); Keir Hart, Lafayette, CO (US); Mark J. Huseman, Broomfield, CO (US)

(72) Inventors: Chris Salvino, Scottsdale, AZ (US); Keir Hart, Lafayette, CO (US); Mark J. Huseman, Broomfield, CO (US)

(21) Appl. No.: 16/698,908

(22) Filed: Nov. 27, 2019

Related U.S. Application Data
(60) Provisional application No. 62/771,953, filed on Nov. 27, 2018.

(51) Int. Cl. A61M 1/04; A61M 39/12

(52) U.S. Cl. CPC A61M 1/04; A61M 2039/2426; A61M 39/12

(57) ABSTRACT

An integrated chest evacuation tube that is specifically configured for being a closed system when inserted and retained inside of a chest cavity provides enhanced benefits over the state of the art. The integrated chest evacuation tube generally includes a valve that is made up of a hollow elastomeric housing having a collar at an output end of the housing which provides an aperture into the housing, a valve input end at an input end of the housing, and an elongated slit at the valve input end. The slit is configured to close together in a spring-like manner when in a closed position. The valve is integrated and at least partially inside of a chest evacuation tube that has a tube inlet port and a tube outlet port. The tube inlet port is adapted to drain fluid and/or air from a human chest cavity. With the collar fixedly attached to the chest evacuation tube at essentially the tube outlet port, the elongated slit is adapted to be opened by a hollow tube when the hollow tube is forcibly pushed through the slit from a direction of the collar.

### CROSS-REFERENCE TO RELATED APPLICATIONS

This application claims priority to and the benefit of U.S. Provisional Patent Application No. 62/771,953 entitled: Chest Tube Valve, filed on Nov. 27, 2018.

### FIELD OF THE INVENTION

The present embodiments are directed to an integrated chest evacuation tube and valve system with some applications being used with a chest tube insertion device.

### DESCRIPTION OF RELATED ART

The lungs are surrounded by a pleural sac made up of two membranes, the visceral and parietal pleurae. The parietal pleura lines the thoracic wall, and the visceral pleura surrounds the lung. The pleural space is a potential space between these two layers of pleurae. It contains a thin layer of serous pleural fluid that provides lubrication for the pleurae and allows the layers of pleurae to smoothly slide over each other during respiration. In abnormal circumstances, the pleural space can fill with air and certain types of fluids not normally present requiring drainage.

In the industrialized world, trauma is the leading cause of death in males under the age of forty. In the United States, chest injuries are responsible for one-fourth of all trauma deaths. Many of these fatalities could be prevented by early recognition of the injury followed by prompt management. Some traumatic chest injuries require quick placement of chest tubes to drain out air and/or fluids (such as blood) from the chest cavity.

Several techniques are currently used to insert a chest tube, each of which involves a relatively lengthy manual procedure that requires knowledge and experience. The most common technique involves surgical preparation and draping at the site of the tube insertion (usually at the nipple level-fifth intercostal space, anterior to the mid-axillary line on the affected side), administering local anesthesia to the insertion site, and making a 2-4 cm vertical incision. A clamp is inserted through the incision and spread, tearing muscle and tissue until a tract large enough to accept a finger is created. Next, the parietal pleura is punctured. One way to puncture the parietal pleura is with the tip of a clamp, and the physician, on occasion, places a gloved finger into the incision to confirm the presence of a (locally) free pleural space. Next, the proximal end of the chest tube 145 (FIG. 1) is advanced through the incision into the pleural space. As the chest tube is inserted, it is sometimes directed posteriorly and superiorly towards the apex of the lung or elsewhere in the chest cavity. The goal is for the chest tube to drain the pleural space of both air and/or fluids such as blood. Accordingly, once the chest tube is appropriately in place to clear air and/or fluids (such as blood, infection, a transudate) from the pleural space, the tube is fixed to the skin with sutures around the tube anchoring the tube to the skin, dressing is applied, and the tube covered with a sterile dressing.

Insertion of a chest tube using this standard technique can require more than 15 minutes to accomplish by a physician, requires extensive medical training to be performed properly and can be extremely painful as it is a difficult area to anesthetize due to the intercostal nerve that runs on the bottom of every rib. Further, while performing the procedure, the physician must attend to the patient receiving the chest tube and thus is precluded from attending to other patients.

FIG. 1A depicts a prior art chest tube insertion gun 100 which functions as a chest tube deployment device described in U.S. Pat. No. 7,811,293. This chest tube insertion gun 100 includes a housing 105, a handle 110 with the trigger 125, a probe tip 130 having a circular cutting tip 135 at the distal end thereof, a circular cross-sectioned cannula 140, and a circular cross sectioned chest tube 145. The circular cutting tip 135 rotates outside of the distal end up to a 90° angle of rotation (rotation angle) from its neutral position before rotating back to its neutral position. The circular cutting tip 135 is also able to rotate a small negative angle from its neutral position in order to retract inside of the distal end of the probe tip 130. The rotation angle works well for the circular cross-sectioned cannula 140.

FIG. 2A illustratively depicts a prior art side view drawing of different chest tube deployment device referred to as an actuator scalpel. Similar to the chest tube insertion gun 100, the actuator scalpel 200 provides a different handle system and the scalpel blade 220 that both rotates and travels outside of the probe tip 208 in a circular path. More specifically, the actuator scalpel comprises a handle body 202, a trigger 204, a probe 206, and a probe tip 208 showing the probe tip housing 212. The trigger 204 depicts finger grips 210 adapted to accommodate the fingers of a human hand (not shown). Shown for reference is the probe housing 211 and the body housing 205. In operation, the actuator scalpel 200 is gripped by an operator's (person's) palm positioned along the top of the handle body 207 with two of their fingers positioned in the finger grips 210 whereby upon squeezing the handle 204 towards the handle body 202, the scalpel 220 is made to move in a cutting motion.

FIG. 2B illustratively depicts a top view of the actuator scalpel 200 next to a prior art cannula 140. The cannula 140 is a linear tube (or in some cases arc-shaped, not shown) that is arranged to slide over the probe tip 208 and cover the probe shaft 206 via a base opening 102 and a distal end opening 104. In practice, with the cannula 140 slid over the probe shaft 206, which essentially covers the probe 206, the actuator scalpel 200 is made to cut a pathway into the chest cavity of the patient whereby the cannula 140 is slid off of the probe tip 208 and thereby deployed into the chest of a patient. Accordingly, the probe 206 serves as a chest tube deployment shaft. The cannula 140 provides a drainage pathway for fluid to escape the patient.

FIG. 3A-3C illustratively depict drawings of a prior art Heimlich valve in different states of operation used with a chest tube. More specifically, FIG. 3A shows a side view of a Heimlich valve 270 in an inactive, or unused, state/configuration. As viewed in conjunction with FIG. 3D, the Heimlich valve 270 has an inlet nozzle 276 that is adapted to press into the end of a pliable hollow tube 282 whereby the actual Heimlich valve 270 is always outside of a tube, which could be a chest tube (not shown), a pliable rubber sleeve 272, and an outlet nozzle 274 adapted to press into the end of a pliable hollow outlet tube 278 that leads to a fluid collection bag 280. FIG. 3B depicts the pliable rubber sleeve 272 closed, i.e., sealed off when air and/or fluid flows into the outlet nozzle 274 thereby preventing air and/or fluid flowing into a human's chest cavity 288. FIG. 3C depicts the pliable rubber sleeve 272 opened to allow air and/or fluid to flow in the proper direction through the inlet nozzle 276, through the Heimlich Valve body, and out the outlet nozzle 274.

FIG. 3D illustratively depicts a drawing of a prior art drainage system with the Heimlich Valve assembly engaged with a human patient. More specifically, a chest tube 145 is inserted into a lung space 286 via an incision 290 in a chest cavity 288 of a human patient/subject 299. The chest tube 145 is connected to a valve 284 that allows an operator (not shown) to open and close passage between the chest tube 145 and the fluid collection bag 280. Connected to the other side of the pigtail valve 284 is an intermediate pliable hollow tube 282 pressed into the inlet nozzle 276 of the Heimlich valve 270. The outlet nozzle 274 of the Heimlich valve 270 is pressed into a pliable hollow outlet tube 278 which is connected to the fluid collection bag 280. Accordingly, there is a direct path between the lung space 286 and the fluid collection bag 280. As can be readily appreciated, fluid and/or air from the fluid collection bag 280 is prevented from back flowing into the subject's/patient 299. Though there are numerous advantages to the present a state-of-the-art Heimlich Valve 270 and the assembly shown in FIG. 3D, applications in an emergency situation can be cumbersome and problematic.

It is to innovations related to this subject matter that the claimed invention is generally directed.

### SUMMARY OF THE INVENTION

The present embodiments are directed to simplification of the state-of-the-art Heimlich valve 270 and Heimlich valve assembly as shown in FIG. 3D by way of an integrated chest evacuation tube and valve.

Certain embodiments of the present invention contemplate an integrated chest tube valve comprising: a collar at an output end of the valve that defines an output aperture; a hollow elastomeric body extending from the collar to an input end, the input end possessing an elongated slit; and a chest evacuation tube possessing an inlet port and an outlet port, the inlet port adapted to drain fluid and/or air from a human chest cavity, the input end is inside of the chest evacuation tube, the collar is fixedly attached to the chest evacuation tube at essentially the outlet port, the elongated slit is in a closed configuration but is adapted to be in an opened configuration when a hollow tube is forcibly pushed through the elongated slit via the collar, the hollow tube is less compliant than the hollow elastomeric body.

Yet other certain embodiments of the present invention contemplate a method comprising: providing an integrated chest evacuation tube valve possessing a hollow elastomeric housing, a collar at an output end of the housing, the collar defining an aperture into the housing the housing extending from the collar to a valve input end, the valve input end possessing an elongated slit that is configured to close together in a spring-like manner when in a closed configuration, the valve input end inside of a chest evacuation tube, the chest evacuation tube possessing a tube inlet port and a tube outlet port; inserting the tube inlet port in a human chest cavity; pushing a rigid tube through the hollow elastomeric housing and through the elongated slit via the collar from the tube outlet port; and after the pushing step, draining fluid and/or air from the human chest cavity from the tube outlet port by way of the rigid tube.

While other certain embodiments of the present invention contemplate an integrated chest evacuation tube valve comprising: a hollow elastomeric housing; a collar at an output end of the housing, the collar defining an aperture into the housing; the housing extending from the collar to a valve input end, the valve input end possessing an elongated slit that is configured to close together in a spring-like manner when in a closed configuration; and a chest evacuation tube possessing an inlet port and an outlet port, the inlet port adapted to drain fluid and/or air from a human chest cavity, the input end is inside of the chest evacuation tube, the collar fixedly attached to the chest evacuation tube at essentially the tube outlet port, the elongated slit adapted to be opened by a hollow tube when the hollow tube is forcibly pushed through the elongated slit from a direction of the collar.

### BRIEF DESCRIPTION OF THE DRAWINGS

FIG. 1 illustratively depicts a prior art drawing chest tube insertion gun;

FIG. 2A illustratively depicts a front isometric view drawing of the prior art actuator scalpel of FIG. 2A;

FIG. 2B illustratively depicts a drawing of a top view of the prior art actuator scalpel of FIG. 2A next to a prior art cannula;

FIG. 3A-3C illustratively depict drawings of a prior art Heimlich Valve in different states of operation used with a chest tube;

FIG. 3D illustratively depicts a drawing of a prior art close drainage system with the pigtail Heimlich Valve assembly engaged with a human patient;

FIGS. 4A-4D illustratively depict different drawing views of a duckbill valve consistent with embodiments of the present invention;

FIGS. 5A and 5B illustratively depict perspective front view drawings of a different valve embodiment with a slit only at the input and consistent with embodiments of the present invention;

FIGS. 6A-6C depict hollow tube embodiments adapted to forcibly open the valve 400 consistent with embodiments of the present invention;

FIGS. 7A and 7B illustratively depict a side view of two different valve embodiments forcibly opened via a hollow tube consistent with embodiments of the present invention;

FIG. 8A illustratively depicts a typical location where an embodiment of the integrated chest evacuation tube valve can be deployed;

FIG. 8B illustratively depicts an integrated chest evacuation tube and valve system being inserted between ribs of a patient/subject consistent with embodiments of the present invention;

FIG. 9A depicts a perspective view drawing of another embodiment of a chest evacuation tube consistent with embodiments of the present invention;

FIGS. 9B and 9C illustratively depict perspective drawings of different placements of a valve embodiment consistent with embodiments of the present invention;

FIG. 9D illustratively depicts a side view drawing of a valve embodiment integrated with the chest cannula consistent with embodiments of the present invention;

FIGS. 10A and 10B illustratively depict drawings of a different integrated chest evacuation tube embodiment, namely a typical chest tube, consistent with embodiments of the present invention;

FIGS. 11A-11F are line drawings illustratively depicting implementation of a chest cannula and chest tube consistent with embodiments of the present invention; and

FIG. 12 illustratively depicts a drawing of an integrated chest tube evacuation and valve system employed in a patient and connected to a fluid collection bag consistent with embodiments of the present invention.

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of situations involving similar uses of an integrated chest evacuation tube and valve system. In what follows, similar or identical structures may (and may not) be identified using identical callouts.

Described herein are embodiments of an integrated chest evacuation tube specifically configured as a closed system (i.e., preventing fluid/air from unintentionally escaping through the chest evacuation tube subjecting medical providers to body fluids) when inserted and retained inside of a chest cavity. The embodiments of the closed-system chest evacuation tube described herein provide enhanced benefits over the present state of the art. For example, the commonly used Heimlich valve permits fluid and/or air to flow out of the body but not in reverse in contrast to the embodiments described below which illustratively describe prevention of unwanted fluid and/or air to flow out of the body. Certain embodiments envision the integrated chest evacuation tube generally including a valve that comprises a hollow elastomeric housing having a collar at an output end of the housing, which provides an aperture into the housing, a valve input end at an input end of the housing, and an elongated slit at the valve input end. The slit is configured to close together in a spring-like manner when in a closed position. The valve is integrated with the chest evacuation tube and, in certain embodiments, is at least partially inside of the chest evacuation tube. The chest evacuation tube has a tube inlet port that is adapted to drain fluid from a human chest cavity. With the collar fixedly attached to the chest evacuation tube at essentially the tube outlet port, the elongated slit is configured to open via a hollow tube forcibly pushing through the slit from the collar.

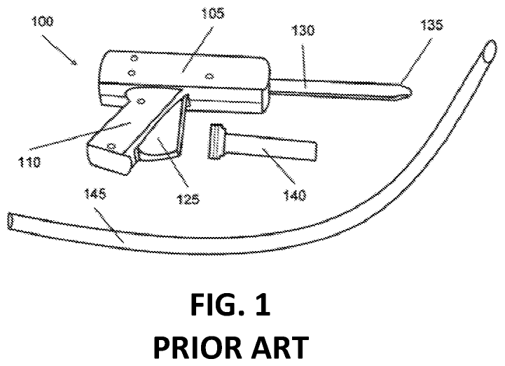

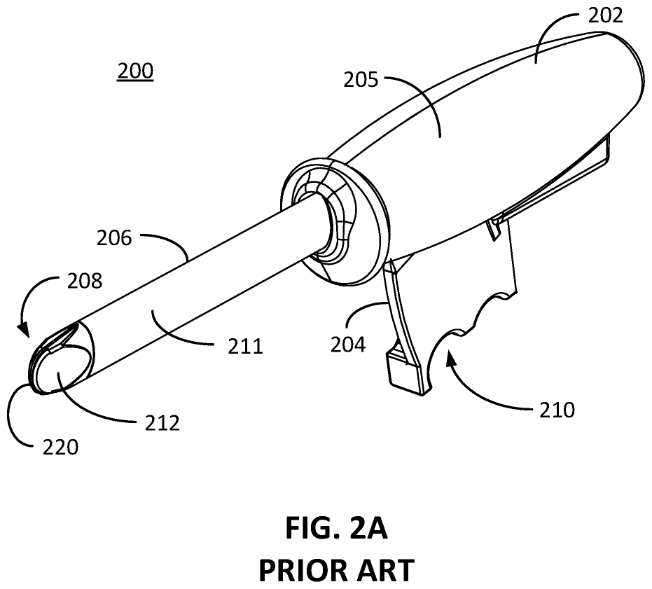

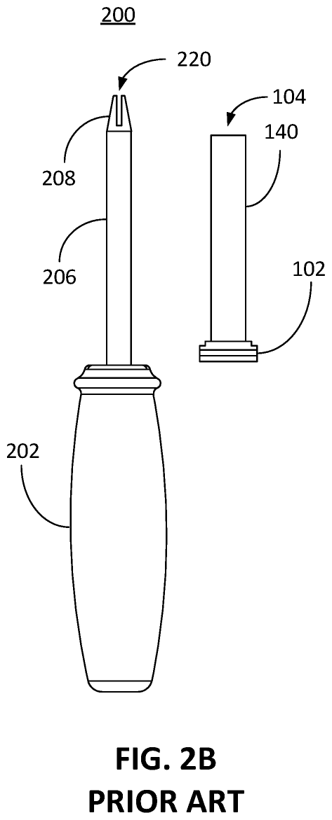

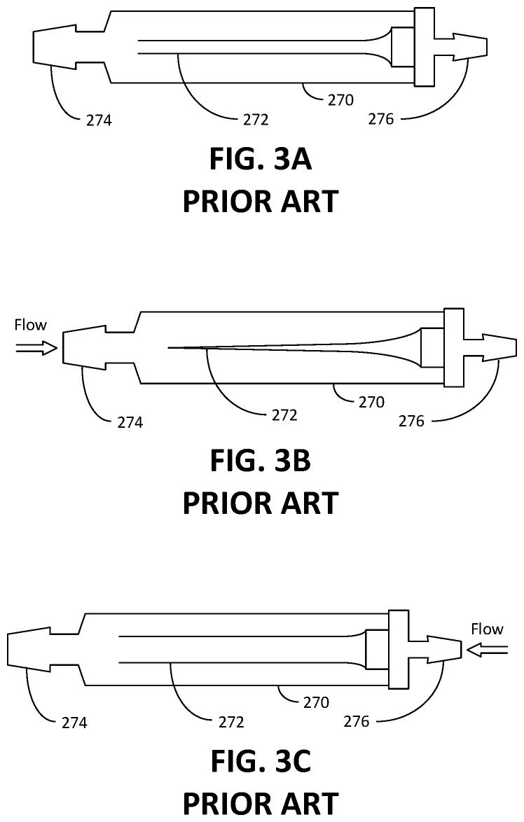

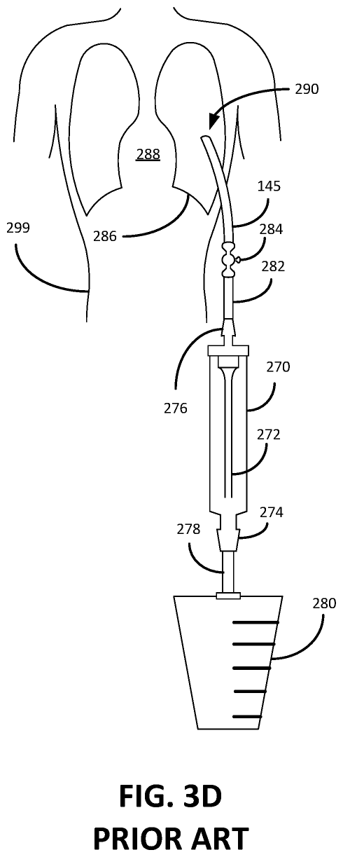

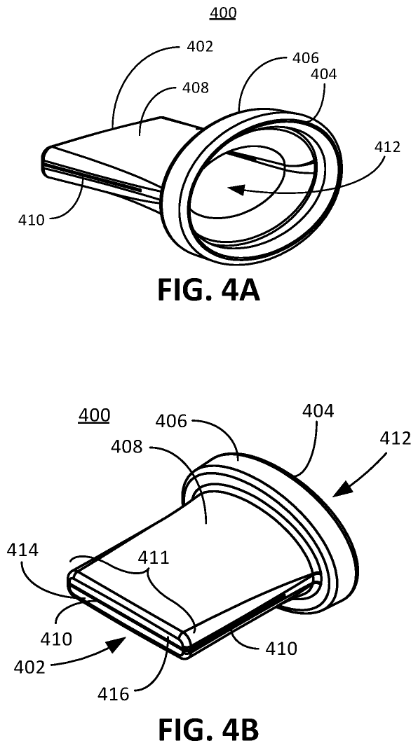

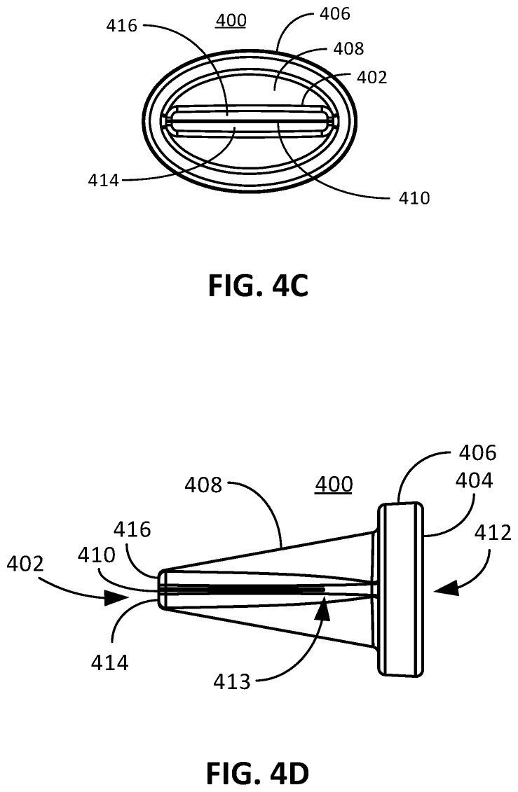

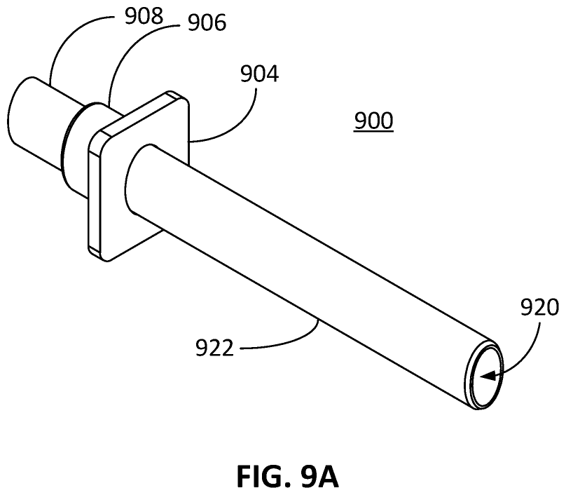

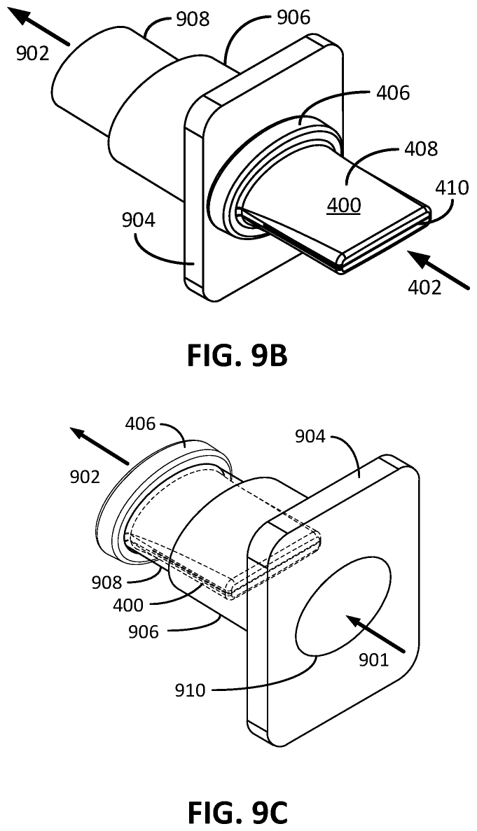

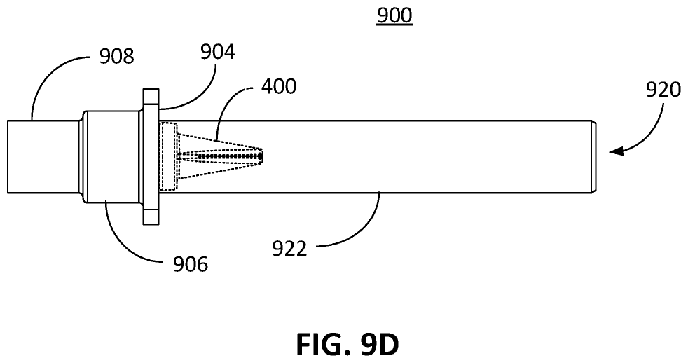

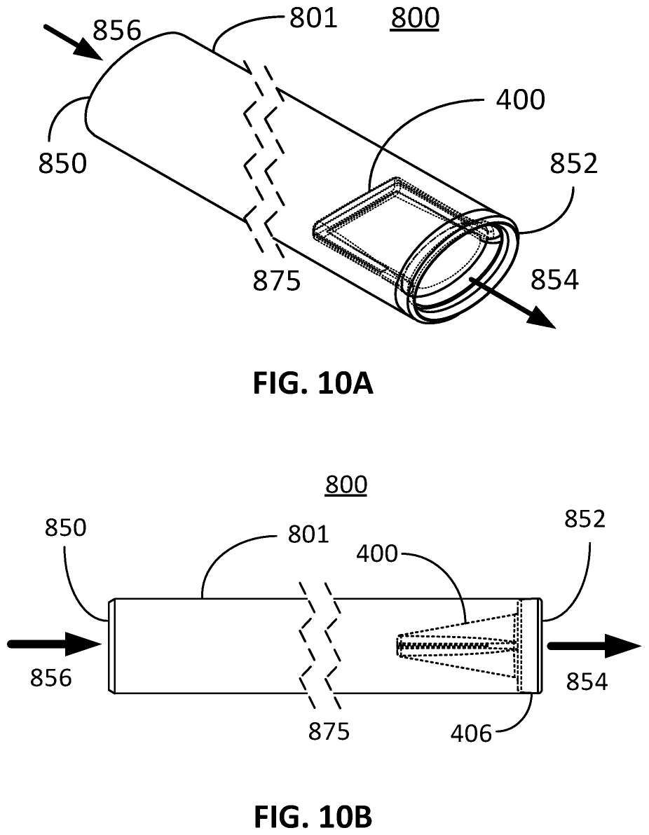

FIGS. 4A-4D illustratively depict different line drawing views of a duckbill valve consistent with embodiments of the present invention. FIG. 4A shows an embodiment of a duckbill valve 400 (or simply the “valve 400”) in a one-quarter perspective view with the output end 404 displayed. The valve 400 comprises a collar 406 (which in some embodiments is referred to as a “ring”), a hollow elastomeric housing/body 408 possessing and outlet port or opening (also referred to herein as an output aperture) 412, depicted by the output aperture arrow 412, and a slit 410 at an input end 402. In the present embodiment, the physical response of the elastomeric housing is to deform when manipulated but to spring back or otherwise return to its original shape when no longer manipulated. The hollow elastomeric housing (body) 408 can be made from a number of elastomeric materials, which exhibit elastic or rubber-like properties, for example a polymer with high elasticity, a low Young's modulus and a high failure strain compared to other materials. Natural rubber, neoprene rubber, buna-s and buna-n are all examples of elastomer species that can be used within the scope and spirit of the present invention. In the present drawing, the valve 400 is in a closed configuration (configuration/position) whereby the elongated slit 410 is shut. This embodiment depicts the collar 406 as elliptically shaped to conform with an elliptically shaped chest evacuation tube (e.g., a chest tube 800 or a chest cannula 900 integrated with the valve, see FIGS. 9A and 10A). Other embodiments contemplate a circular shaped collar 406 configured to conform with a circular shaped chest tube 145 or circular cannula 140 (see FIG. 1). Some certain embodiments envision the collar 406 being rigid while other embodiments envision the collar 406 being compliant but more rigid than the valve body 308 (which in certain embodiments is accomplished by way of a different material or the same material that is thicker and therefore stiffer).

FIG. 4B illustratively depicts a perspective drawing showing the input end 402 of the valve 400 in a closed configuration consistent with embodiments of the present invention. The elongated slit 410 extends across the width 411 of the front of the valve 402 (input end) and extends along the hollow elastomeric housing 408 from the input end 402 to the collar 406. Optionally, the elongated slit 410 extends somewhere between the input end 402 and the collar 406. For purposes of explanation, the material at the input end 402 above the slit 410 is referred to herein as the upper lip 416, and the material at the input end 402 below the slit 410 is referred to herein as the lower lip 414. The upper lip 416 and the lower lip 410 remain clamped, or otherwise closed together, in a spring-like manner thereby preventing any fluid or air to pass through the valve input end 402 unless forcibly opened.

FIG. 4C illustratively depicts a front view drawing of the valve 400 showing the input end 402 in a closed configuration consistent with embodiments of the present invention. The elongated slit 410 stretches across the entire width of the input end 402 and is defined by the interface between the upper lip 416 and the lower lip 414. The hollow elastomeric housing/body 408 and the elliptical shaped collar 406 are shown for reference.

FIG. 4D illustratively depicts a side view drawing of the valve 400 and a closed configuration consistent with embodiments of the present invention. In the present embodiment, the slit 410 extends only partway between the input end 402 and the collar 406 at the location where the arrow 413 is pointing. As previously discussed, other embodiments envision the slit 410 extending all the way to the collar 406. As shown, the hollow elastomeric housing 408 is sloped between the collar 406 and the input end 402. Certain embodiments envision the hollow elastomeric housing having a curved profile (either concave or convex) between the collar 406 and the input end 402.

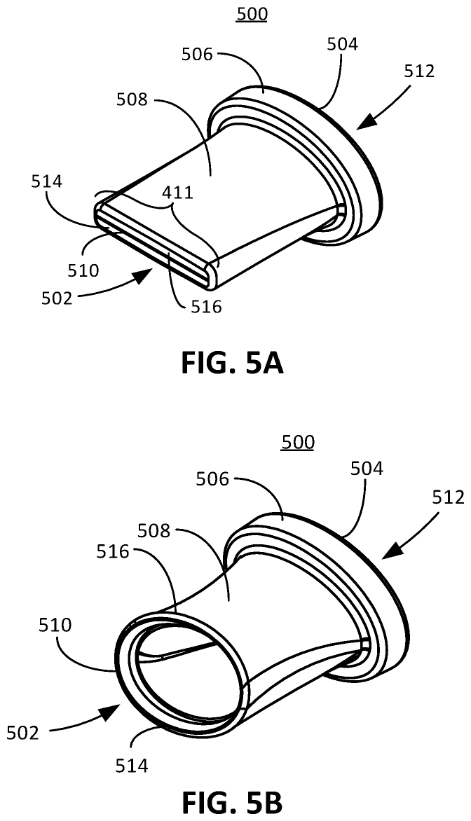

FIGS. 5A and 5B illustratively depict isometric front view line drawings of a different valve embodiment with a slit only at the input end consistent with embodiments of the present invention. As shown in FIG. 5A, the valve 500 possesses a slit 510 that extends across the width 411 of the input end 502. In this embodiment, the slit 510 does not extend along the side of the hollow elastomeric body 508 as in the previous embodiment. The slit 510 is defined by the interface between the upper lip 516 and the lower lip 514. The collar 506 can be the same as the collar previously described having in outlet end 504 and an aperture 512 (or opening) providing an outlet port for the valve 500. The valve depicted in FIG. 5A is in a closed configuration with the upper lip 516 and the lower lip 514 pressed together in a spring-like manner by the spring force generated from the elastomeric material of the valve 500.

FIG. 5B illustratively depicts a perspective front view drawing of the valve 500 in an open configuration whereby the upper lip 516 and the lower lip 514 are forced apart through physical manipulation thereby providing a clear path between the input end 502 and the output end 504. Accordingly, when the slit 510 is forced to be in an open configuration, fluid and/or air is free to pass through the valve 500.

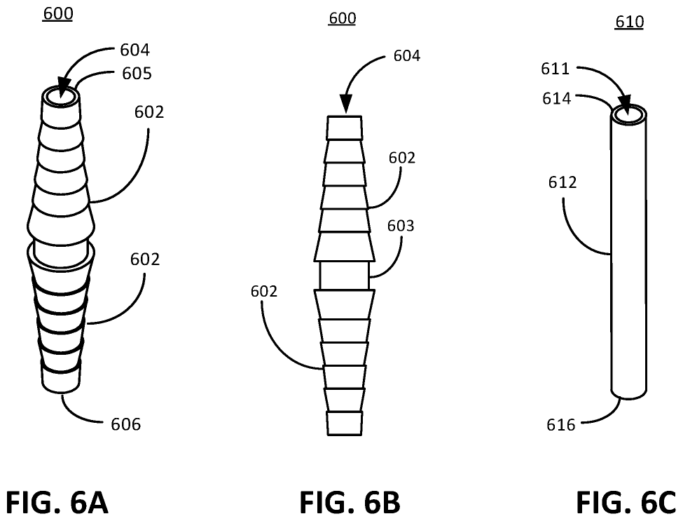

With continued reference to FIG. 4A-4D, as mentioned the valve 400 remains in a closed configuration because of the elastomeric material forcing the upper lip 416 and the lower lip 414 together by way of a spring-like force unless forced open. FIGS. 6A-6C depict hollow tube embodiments that can be used to forcibly open the valve 400 (and valve 500) consistent with embodiments of the present invention. FIGS. 6A and 6B illustratively depict a perspective drawing and a side view drawing of a barbed tube 600, respectively. The barbs 602 (labeled twice) help lock or otherwise retain the barbed tube 600 either in the valve 400 or a flexible connecting tube (not shown). Further, as shown, the barbs 602 are in opposing directions on either side of the center location 603 and are either circular or elliptical (or some other tube matching shape) depending on the tube shape. The barbed tube 600 is a hollow tube that provides an unobstructed, and clear, pathway 604 through the tube 600 between an inlet aperture 605 and an outlet aperture 606. Certain embodiments envision the barbed tube 600 being rigid while other embodiments envision the barbed tube 600 simply being stiff enough to overcome spring-like force, keeping the upper lip 416 and the lower lip 414 in a closed configuration.

FIG. 6C depicts a perspective drawing of a smooth rigid hollow tube 610 embodiment that provides an unobstructed clear pathway 611 that passes through the tube 610 between an inlet aperture 614 and an outlet aperture 616. As shown, the rigid hollow tube 610 has a smooth outer surface 612. Certain embodiments envision the inner surface being smooth for both the barbed tube 600 and the smooth tube 610 embodiments. As with the barbed tube 600, certain embodiments envision the smooth tube 610 being rigid or optionally simply being stiff enough to overcome the spring-like force keeping the upper lip 416 and the lower lip 414 in a closed position. The tubes 600 and 610 can be composes of a stiff polymer, metal, or other rigid material know to those skilled in the art.

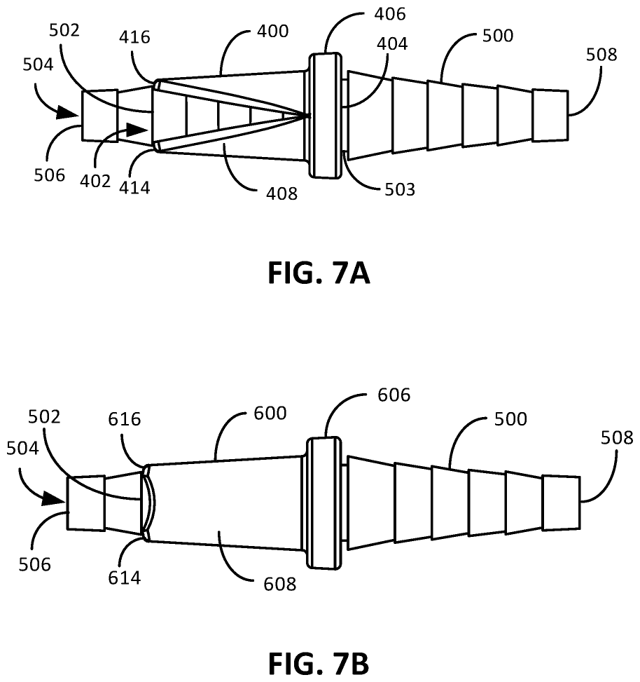

FIGS. 7A and 7B illustratively depict a side view of two different valve embodiments forcibly opened via a hollow tube consistent with embodiments of the present invention. As shown in FIG. 7A, a barbed tube 600 has been forcibly inserted/pushed through the output end 404 of the valve 400 via the collar 406 and through the input end 402 thereby spreading the upper lip 416 and the lower lip 414 apart to open the valve 400. In other words, a surgeon or operator is envisioned to hold the valve 400 with one hand while inserting the barbed tube 600 (or some other tube) through the output end 404 to open the valve 400 (forcing the valve 400 open) thereby creating a clear pathway from the inlet end 402 to the outlet end 404. More specifically, the elastomeric valve body 408 complies with the barbed tube 600, and due to the spring force of the elastomeric material of the upper lip 416 and the lower lip 414, the valve 400 is sprung open conforming to the outer surface of the barbed tube 600. In this embodiment, the barbed tube 600 is inserted in the valve 400 approximately up to the center location 603 whereby a lip of a barb 602 locks against the edges of the upper lip 416 and the lower lip 414. Because the outer surface of each segment of the barbed tube 600 is sloped in increasing diameter from a previously barbed segment, the barbed tube 600 is essentially retained or essentially locked in position unless manipulated by force to overcome being retained by the valve 400. Certain embodiments envision the barbed tube 600 conforming to the shape of the collar 406. For example, if the collar 406 is elliptical then the unobstructed clear pathway 604 is also elliptical. Embodiments of the present invention envision inserting the smooth hollow tube 610 in the output end of the valve 400 in a similar way as a described in the barbed tube embodiment 600 without departing from the scope and spirit of the present invention.

FIG. 7B illustratively depicts the barbed tube 600 being pushed through the collar 506 of the valve embodiment 500 consistent with embodiments of the present invention. Like FIG. 7A, the upper lip 516 and the lower lip 514 are spread apart to the edge of the second barb 602 (which in this embodiment fits around the circumference of the tube 600) thereby closing around or otherwise conforming to the barbed tube 600 by way of the spring-force provided by the elastomeric housing 508. Embodiments of the present invention envision inserting the smooth hollow tube 610 in the output end of the valve 500 in a similar way as a described in the barbed tube embodiment 600 without departing from the scope and spirit of the present invention.

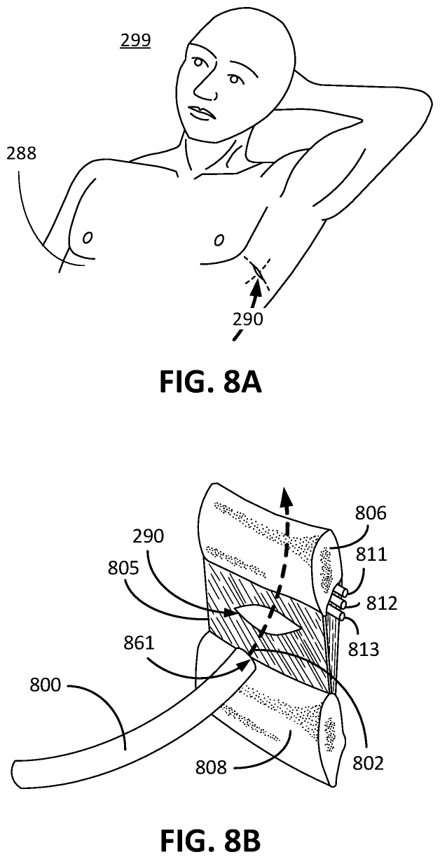

FIG. 8A illustratively depicts a typical location in a line drawing of a person where an embodiment of the integrated chest evacuation tube valve can be deployed. As shown, the patient/subject 299 is marked with a dashed-X 290 pointing to a typical location at the fifth rib (under the armpit) where a chest evacuation tube (e.g., chest tube 880 or chest cannula 900, shown in FIGS. 9A and 10A, for example) can be deployed. The dashed-X 290 resides soundly in a good place to access the internal locations of the chest cavity 288 wherein fluid/air buildup can occur due to trauma, for example.

FIG. 8B illustratively depicts one embodiment of the chest evacuation tube 801 integrated with a valve embodiment being inserted between ribs of a patient/subject consistent with embodiments of the present invention. Here, an incision 290 is made via the actuator scalpel 200 (not shown in this figure) in the intercostal muscles 805 between an upper rib 806 and a lower rib 808. Certain embodiments envision the integrated chest evacuation tube and valve 800 (which in certain embodiments includes a cannula, a curved cannula or a chest tube integrated with the valve embodiment of the present invention) being oval in cross-section of at least a curved polymer tube portion, in order to fit more effectively between the ribs 806 and 808, while providing greater volume of built-up liquid/air to drain out from the chest cavity 288. For reference, the intercostal artery 812 and the intercostal vein 811 are shown.

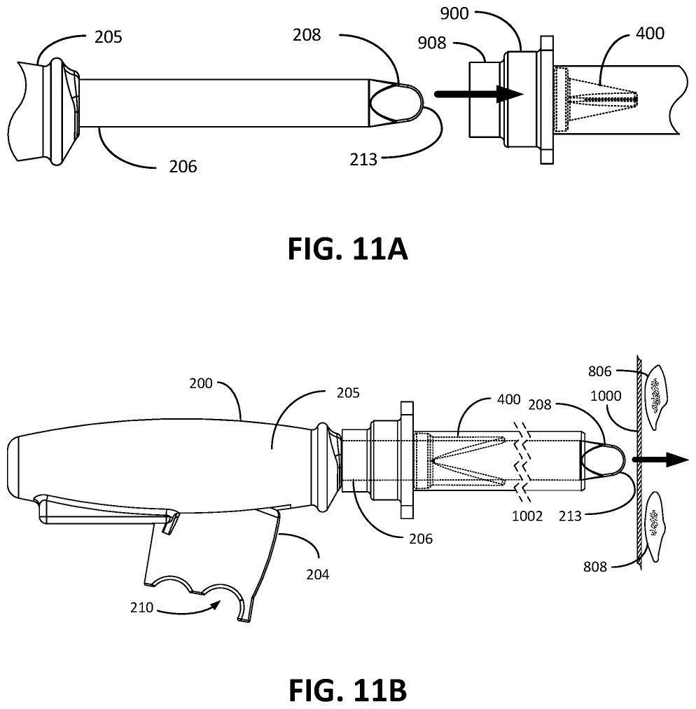

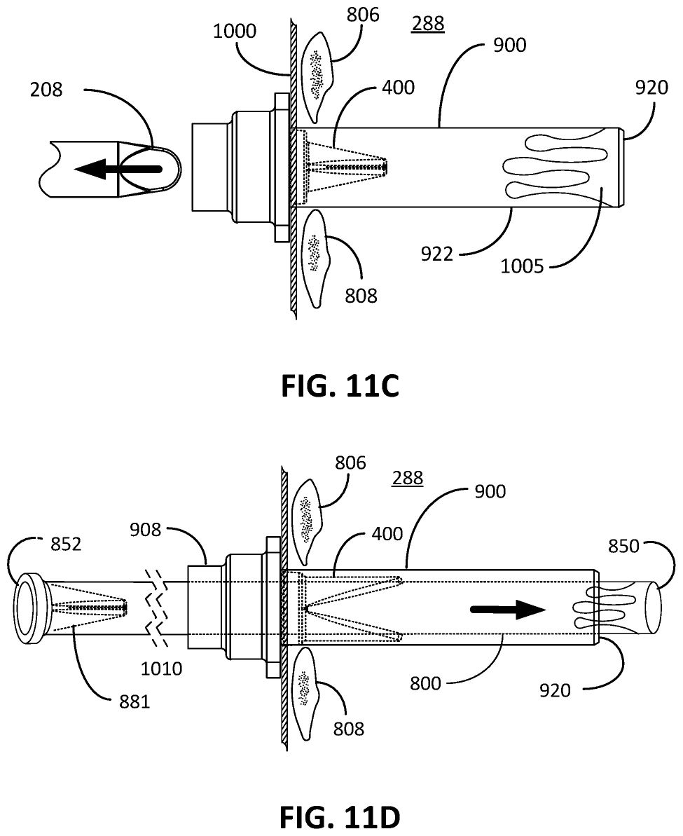

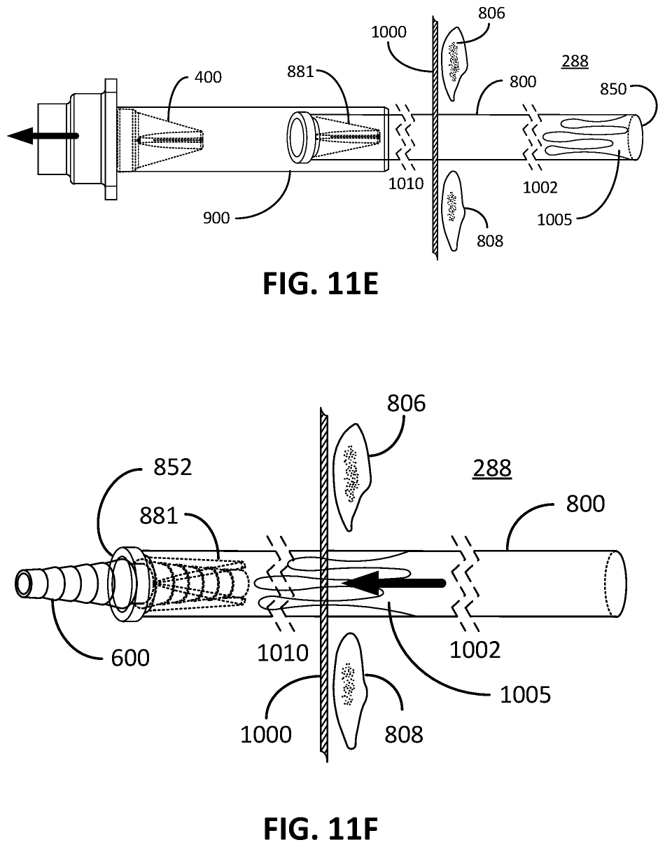

FIG. 9A depicts an isometric drawing of a chest tube cannula embodiment consistent with embodiments of the present invention. As shown, the cannula 900 (which can function as a chest tube) generally possesses a distal end 920 that serves as an inlet port, a proximal end 908 that terminates in an outlet port, and a flexible tube member 922. More specifically, the proximal end 908 (where the proximal aperture 908, or opening, is located) is adapted to slide over a tip 208 of the handheld actuator scalpel 200. The distal end 920 is configured to penetrate into the chest cavity of a recipient via an incision, which in certain embodiments is a human subject (but is not so limited to being a human subject). A distal tube opening 920 (also referred to as a distal tube aperture) in the cannula 900 provides an entryway into the cannula 900 through which fluids from the chest cavity can exit. An integrated valve and chest tube 800, or other device, can be inserted through the cannula 900 and into the chest cavity 288. Certain embodiments envision the cannula 900 being pliable to conform and bend when inserted in the chest cavity for improved maneuverability and comfort of the recipient. Also shown is a stop plate 904 adapted to cover an incision 290 in the subject's chest, which can be used as a platform to suture the cannula 900 to the subject's skin (see FIG. 11C). The stop plate 904 is further adapted to help or control body fluids from leaking out of the incision 290 at the subject's chest cavity. The cannula 900 can further comprise a rigid or semi-rigid grip collar 906 that an operator can hold and manipulate with his or her fingers. Some embodiments contemplate the tube 922 and the collar 906 being a unitary structure.

FIGS. 9B and 9C illustratively depict perspective drawings of optional valve embodiment placements in a cannula consistent with embodiments of the present invention. As shown in FIG. 9B, the duckbill valve 400 is integrated with the stop plate 904 whereby the valve 400 resides inside of the cannula tube 922 with the input end 410 of the valve 400 arranged to point towards the cannula's inlet port 920. The collar 406 essentially conforms to the shape of the opening 910 and provides a continued pathway via the opening 910 (FIG. 9C). The cannula tube 922 is not shown in FIGS. 9B and 9C for ease of viewing and explanation, however, in certain embodiments, the cannula tube 922 would cover the valve 400. FIG. 9C depicts yet a different embodiment of an integrated chest cannula and valve system wherein the collar 406 is fixedly attached to the proximal end/aperture 908. As shown, the input end 410 of the valve 400 is arranged to point towards the cannula inlet port 920. Based on the images, it should be readily apparent that the valve 400 prevents flow in the direction of the arrow 902 when in a closed configuration.

FIG. 9D illustratively depicts a side view line drawing of a valve embodiment integrated with the chest cannula consistent with embodiments of the present invention. The valve 400 is depicted in dashed lines because it is inside of the hollow cannula tube 922. This particular side view embodiment is consistent with FIG. 9B. As shown, the valve 400 will block any movement of fluid and/or air through the inlet port 920.

FIGS. 10A and 10B illustratively depict line drawings of a different integrated chest evacuation tube embodiment, namely a typical chest tube, consistent with embodiments of the present invention. As shown in FIG. 10A, the chest tube 801 (independent of the valve 400) is defined as a long flexible tube between an inlet port 850 and an outlet port 852. The jagged dashed-line break 875 indicates that the chest tube 801 is (much) longer than is shown. Flow is intended to move in the direction of the arrows (arrow 856 through the inlet port 850 and arrow 854 through the outlet port 852). In the present embodiment, the valve 400 is fixedly attached essentially at the outlet port 852, however other embodiments envision a valve embodiment being fixedly attached in another location along the length of the chest tube 801 between the inlet port 850 and the outlet port 852. Here, the integrated valve and chest tube 800 are used interchangeably with the term chest tube.

FIG. 10B illustratively depicts a side view line drawing of the embodiment of FIG. 10A again with the jagged dashed-line break 875 indicating that the chest tube 800/801 is considerably longer than depicted in the figure. The arrows 856 and 854 indicate the direction of the flow when the valve 400 is open.

FIGS. 11A-11F are drawings illustratively depicting deployment of a chest cannula in conjunction with a chest tube consistent with embodiments of the present invention. In these embodiments, their order of operation are as shown. In practice, the embodiment shown depicts the cannula 900 integrated with the valve 400 being slid over the probe shaft 206 in the direction of the arrow of FIG. 11A. More specifically, the probe tip 208 is inserted inside of the valve integrated cannula 900 by way of the inlet port 908 and through aperture 412 defined by the collar 406 of the valve 400.

FIG. 11B illustratively shows a side view of the actuator scalpel 200 essentially wearing the valve integrated cannula 900 over the probe shaft 206. The probe tip 208 extends out the inlet port 920 of the cannula 900 in order for the scalpel blade 213 to freely cut through human skin 1000 of a chest wall as shown by the arrow. The upper rib 806 and the lower rib 808 from FIG. 8B are shown here for reference. The actuator scalpel 200 is held by a human hand (not shown) that wraps around the housing and the finger grips 210 of the trigger 204. When squeezed, the scalpel blade 213 extends from the probe tip 208 in a cutting/slicing action thereby cutting an incision 290 through the skin 1000.

As illustratively depicted in FIG. 11C, once the valve integrated cannula 900 is inserted in the chest cavity 288, the actuator scalpel 200 is withdrawn from the cannula 900 as shown by the arrow. This is considered deployment of valve integrated cannula 900 (in the chest cavity 288). As shown, the valve 400 is in a closed configuration preventing fluid and/or air 1005 (at the inlet port 920) from escaping through the outlet port of the cannula 900. Certain embodiments envision the cannula 900 being sutured in place to the skin 1000 in the arrangement as shown thereby serving as the chest evacuation tube (if so desired). There are applications for using just the cannula 900 as the chest evacuation tube, such as in time sensitive emergencies. In this scenario, FIGS. 11D-11E are not carried out and FIG. 11F is modified for the cannula 900.

FIG. 11D depicts the continued process of inserting a chest tube 800 through the cannula 900 to essentially replace the cannula 900 as the chest evacuation tube. More specifically, the chest tube 800 is threaded through the outlet port of the cannula 900, through the valve 400 and out the cannula outlet port 920 as shown by the arrow. In the present embodiment, the chest tube 801 has an integrated valve 881 (which in this case is a duck bill valve) that is fixedly attached to the chest tube outlet port 852. The present embodiment depicts the chest tube inlet port 850 extending through the cannula outlet port 920 into the chest cavity 288. The chest tube 800 is shown with a broken line 1010 indicating that the chest tube 800 is longer than is shown in FIG. 11D.

With continued reference to the deployment steps, FIG. 11E shows the cannula 900 being withdrawn from the chest cavity 288 by sliding the cannula 900 over the chest tube 800 as shown by the arrow. Broken line 1002 indicates that the chest tube 800 is longer than shown. Note that the chest tube valve 881 is in a closed configuration. The chest tube 800 can be sutured or otherwise attached to the skin 1000.

FIG. 11F shows the insertion of the barbed tube 600 forcing the chest tube valve 881 in an opened configuration thereby permitting air and/or body fluid 1005 to escape via the outlet port 852 (see arrow), presumably into a fluid collection bag or some other receptacle.

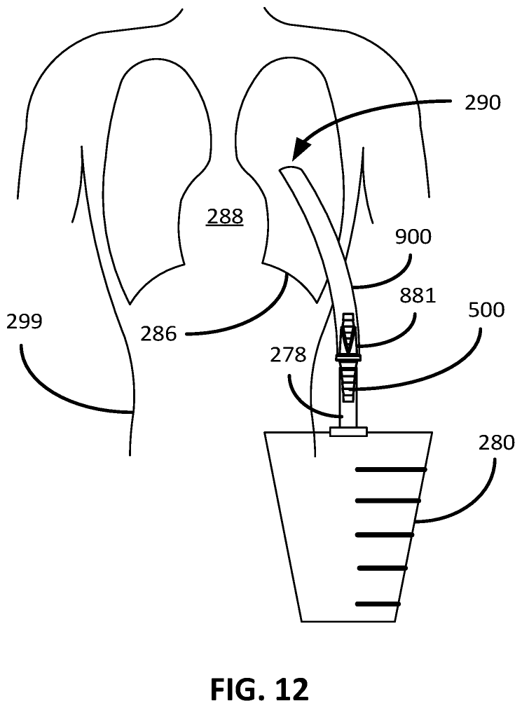

FIG. 12 illustratively depicts a drawing of an integrated chest tube evacuation and valve system employed in a patient and connected to a fluid collection bag consistent with embodiments of the present invention. As shown, the chest tube 800 is inserted into a chest cavity 288 of a patient 299 through an incision 290. Fluid 1005 collecting in the lung space 286 can be drained through the chest tube 800 via the valve 881 that is opened by the barbed tube 600 and into the fluid collection bag/receptacle 280. The other end of the barbed tube 600 is pressed into a flexible tube 278 that is connected to the fluid collection bag 280.

With the present description in mind, some embodiments of the present invention contemplate:

An integrated chest tube valve 400 comprising: a collar 406 at an output end 404 of the valve 400 that defines an output aperture 412; a hollow elastomeric body 408 extending from the collar 406 to an input end 402, the input end 402 possessing an elongated slit 410; and a chest evacuation tube 801 possessing an inlet port 850 and an outlet port 852, the inlet port 850 adapted to drain fluid and/or air 1005 from a human chest cavity 288, the input end 402 is inside of the chest evacuation tube 801, the collar 406 is fixedly attached to the chest evacuation tube 801 at essentially the outlet port 852, the elongated slit 410 is in a closed configuration but is adapted to be in an opened configuration when a hollow tube 600 is forcibly pushed through the elongated slit 410 via the collar 406, the hollow tube 600 is less compliant than the hollow elastomeric body 408.

The integrated chest tube valve 400 embodiment further envisioning wherein the hollow tube 600 extends into the valve 406 less than 1 inch from the input end 402.

The integrated chest tube valve 400 embodiment further envisioning wherein the elongated slit 410 seals the outlet port 852 of fluid and/or air coming from the chest evacuation tube 801 inlet port 850 when the elongated slit 410 is in the closed configuration.

The integrated chest tube valve 400 embodiment further envisioning wherein the hollow tube 600 further possesses barbs 602 adapted to retain the hollow tube 600 in an engaged relationship with the elongated slit 410.

The integrated chest tube valve 400 embodiment further envisioning wherein the elongated slit 410 is formed from an upper lip 416 and a lower lip 418, the upper lip 416 and the lower lip 418 are arranged to close together in a spring-like manner when in the closed configuration.

The integrated chest tube valve 400 embodiment further envisioning wherein the hollow tube 600 possesses a tube inlet orifice 605 and a tube outlet orifice 606, the tube inlet orifice 605 extends through the input end 402 and the tube outlet orifice 606 stays outside of the outlet port 852 of the chest evacuation tube 801. This could additionally be wherein the tube outlet orifice 605 provides a direct passage 604 from the chest evacuation tube 801 to a fluid collecting receptacle 280 and further wherein the fluid collecting receptacle 280 is forms a physically fixed connection with the chest evacuation tube 801.

The integrated chest tube valve 400 embodiment further envisioning wherein the collar 406 is more rigid than the hollow elastomeric body 408.

The integrated chest tube valve 400 embodiment further envisioning wherein the chest evacuation tube 801 has an oval cross-section and the collar 406 is oval to match the oval cross-section.

The integrated chest tube valve 400 embodiment further envisioning wherein the chest evacuation tube 801 is a chest tube or wherein the chest evacuation tube 801 is a chest cannula 900 and further wherein the chest cannula 900 is adapted to receive a chest tube 801 via a cannula outlet port 909.

Other embodiments contemplate an integrated chest evacuation tube valve 400 comprising: a hollow elastomeric housing 408; a collar 406 at an output end 404 of the housing 408, the collar 406 defining an aperture 412 into the housing 408; the housing 408 extending from the collar 406 to a valve input end 402, the valve input end 402 possessing an elongated slit 410 that is configured to close together in a spring-like manner when in a closed configuration; and a chest evacuation tube 801 possessing an inlet port 850 and an outlet port 852, the inlet port 850 adapted to drain fluid and/or air 1005 from a human chest cavity 288, the input end 402 is inside of the chest evacuation tube 801, the collar 406 fixedly attached to the chest evacuation tube 801 at essentially the tube outlet port 852, the elongated slit 410 adapted to be opened by a hollow tube 600 when the hollow tube 600 is forcibly pushed through the elongated slit 410 from a direction of the collar 406.

The integrated chest evacuation tube valve 400 embodiment further contemplating wherein the hollow tube 410 extends into the chest evacuation tube 801 far enough to hold the elongated slit 410 in an opened configuration.

The integrated chest evacuation tube valve 400 embodiment further contemplating wherein the elongated slit 410 seals the outlet port 852 of fluid and/or air 1005 coming from the chest evacuation tube 801 inlet port 850 when the elongated slit 410 is not opened by the hollow tube 600.

The integrated chest evacuation tube valve 400 embodiment further contemplating wherein the hollow tube 600 has an outer diameter that is within 75% of an inner diameter of the chest evacuation tube 801.

The integrated chest evacuation tube valve 400 embodiment further contemplating wherein the hollow tube 600 possesses a tube inlet orifice 605 and a tube outlet orifice 606, the tube inlet orifice 605 extends through the input end 402 while the tube outlet orifice 606 never goes inside of the chest evacuation tube 801. The embodiment can additionally be wherein the outlet orifice 606 provides a direct passage from the chest evacuation tube 801 to a fluid collecting receptacle 280 and wherein the fluid collecting receptacle 280 is adapted to form a physically fixed connection with the chest evacuation tube 801.

The integrated chest evacuation tube valve 400 embodiment further contemplating wherein the chest evacuation tube 801 is a chest tube.

The integrated chest evacuation tube valve 400 embodiment further contemplating wherein the chest evacuation tube 801 is a chest cannula 900.

The integrated chest evacuation tube valve 400 embodiment further contemplating wherein the cannula 900 is adapted to receive a chest tube 801 via the outlet port 852.

Certain other embodiments contemplate a method comprising: providing an integrated chest evacuation tube valve 400 possessing a hollow elastomeric housing 408, a collar 406 at an output end 404 of the housing 408, the collar 406 defining an aperture 412 into the housing 408 the housing 408 extending from the collar 406 to a valve input end 402, the valve input end 402 possessing an elongated slit 410 that is configured to close together in a spring-like manner when in a closed configuration, the valve input end 402 inside of a chest evacuation tube 801, the chest evacuation tube 801 possessing a tube inlet port 850 and a tube outlet port 852; inserting the tube inlet port 850 in a human chest cavity 288; pushing a rigid tube 600 through the hollow elastomeric housing 408 and through the elongated slit 410 via the collar 406 from the tube outlet port 852; and after the pushing step, draining fluid and/or air 1005 from the human chest cavity 288 from the tube outlet port 852 by way of the rigid tube 600.

The method can further comprising connecting a fluid collecting receptacle 280 with the chest evacuation tube 801, the fluid collecting receptacle 280 collecting fluid and/or air 1005 from the chest cavity 288 by way of the tube outlet port 852.

The method can additionally comprising pushing the rigid tube 600 through the elongated slit 410 up to a barb 602 on the rigid tube 600, the barb 602 retaining the rigid tube 600 in essentially a fixed position thereby spreading and holding apart two lips 416 and 418 that comprise the elongated slit 410.

The above embodiments are not intended to limit the scope of the invention whatsoever because many more embodiments are easily conceived within the teachings and scope of the instant specification.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, though an integrated chest evacuation tube valve system is depicted by example as a chest cannula 900 and the chest tube 800, other embodiments could equally be used while still maintaining substantially the same functionality without departing from the scope and spirit of the present invention. Another example can include providing various other valve systems that function in the same way directed to a chest evacuation tube without departing from the scope and spirit of the present invention. Though air and fluid are envisioned as two separate compositions that can escape through the tube or tunnel created by the integrated chest evacuation tube valve embodiments, from a physics point of view air is also considered a fluid, hence, if fluid is simply used to define compositions escaping through the integrated chest evacuation tube valve system, it is reasonable to consider that fluid includes air. Yet another example can include variations of a chest evacuation tube, such as using different kinds of structures in the chest evacuation tube including perforation holes, raised elements such as ribs, or other features apparent within the scope and spirit of the present invention. Further, the term “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

What is claimed is:

1. An integrated chest tube valve comprising:
a collar at an output end of the valve that defines an output aperture;
a hollow elastomeric body extending from the collar to an input end, the input end possessing an elongated slit; and
a chest evacuation tube possessing an inlet port and an outlet port, the inlet port adapted to drain fluid and/or air from a human chest cavity, the input end is inside of the chest evacuation tube, the collar is fixedly attached to the chest evacuation tube at essentially the outlet port, the elongated slit is in a closed configuration but is adapted to be in an opened configuration when a hollow tube is forcibly pushed through the elongated slit via the collar, the hollow tube is less compliant than the hollow elastomeric body.

2. The integrated chest tube valve of claim 1 wherein the elongated slit seals the outlet port of fluid and/or air coming from the chest evacuation tube inlet port when the elongated slit is in the closed configuration.

3. The integrated chest tube valve of claim 1 wherein the hollow tube further possesses barbs adapted to retain the hollow tube in an engaged relationship with the elongated slit

4. The integrated chest tube valve of claim 1 wherein the elongated slit is formed from an upper lip and a lower lip, the upper lip and the lower lip are arranged to close together in a spring-like manner when in the closed configuration.

5. The integrated chest tube valve of claim 1 wherein the hollow tube possesses a tube inlet orifice and a tube outlet orifice, the tube inlet orifice extends through the input end and the tube outlet orifice stays outside of the outlet port of the chest evacuation tube.

6. The integrated chest tube valve of claim 6 wherein the tube outlet orifice provides a direct passage from the chest evacuation tube to a fluid collecting receptacle.

7. The integrated chest tube valve of claim 7 wherein the fluid collecting receptacle is forms a physically fixed connection with the chest evacuation tube.

8. The integrated chest tube valve of claim 1 wherein the collar is more rigid than the hollow elastomeric body.

9. The integrated chest tube valve of claim 11 wherein the chest evacuation tube is a chest cannula.

10. An integrated chest evacuation tube valve comprising:
a hollow elastomeric housing;
a collar at an output end of the housing, the collar defining an aperture into the housing;
the housing extending from the collar to a valve input end, the valve input end possessing an elongated slit that is configured to close together in a spring-like manner when in a closed configuration; and
a chest evacuation tube possessing an inlet port and an outlet port, the inlet port adapted to drain fluid and/or air from a human chest cavity, the input end is inside of the chest evacuation tube,
the collar fixedly attached to the chest evacuation tube at essentially the tube outlet port, the elongated slit adapted to be opened by a hollow tube when the hollow tube is forcibly pushed through the elongated slit from a direction of the collar.

11. The integrated chest evacuation tube valve of claim 10 wherein the hollow tube extends into the chest evacuation tube far enough to hold the elongated slit in an opened configuration.

12. The integrated chest evacuation tube valve of claim 10 wherein the elongated slit seals the outlet port of fluid and/or air coming from the chest evacuation tube inlet port when the elongated slit is not opened by the hollow tube.

13. The integrated chest evacuation tube valve of claim 10 wherein the hollow tube possesses a tube inlet orifice and a tube outlet orifice, the tube inlet orifice extends through the input end while the tube outlet orifice never goes inside of the chest evacuation tube.

14. The integrated chest evacuation tube valve of claim 13 wherein the outlet orifice provides a direct passage from the chest evacuation tube to a fluid collecting receptacle and wherein the fluid collecting receptacle is adapted to form a physically fixed connection with the chest evacuation tube.

15. The integrated chest evacuation tube valve of claim 10 wherein the hollow tube has an outer diameter that is within 75% of an inner diameter of the chest evacuation tube.

16. The integrated chest evacuation tube valve of claim 10 wherein the chest evacuation tube 801 is a chest cannula.

17. The integrated chest evacuation tube valve of claim 10 wherein the cannula 900 is adapted to receive a chest tube 801 via the outlet port.

18. A method comprising:
providing an integrated chest evacuation tube valve possessing a hollow elastomeric housing, a collar at an output end of the housing, the collar defining an aperture into the housing the housing extending from the collar to a valve input end the valve input end possessing an elongated slit that is configured to close together in a spring-like manner when in a closed configuration, the valve input end inside of a chest evacuation tube, the chest evacuation tube possessing a tube inlet port and a tube outlet port;
inserting the tube inlet portion a human chest cavity;
pushing a rigid tube through the hollow elastomeric housing and through the elongated slit via the collar from the tube outlet port; and
after the pushing step, draining fluid and/or air from the human chest cavity from the tube outlet port by way of the rigid tube.

19. The method of claim 18 further comprising connecting a fluid collecting receptacle with the chest evacuation tube, the fluid collecting receptacle collecting fluid and/or air from the chest cavity by way of the tube outlet port.

20. The method of claim 18 further comprising pushing the rigid tube through the elongated slit up to a barb on the rigid tube, the barb retaining the rigid tube in essentially a fixed position thereby spreading and holding apart two lips and that comprise the elongated slit.
